(19) United States
(12) Patent Application Publication
(10) Pub. No.: US 2017/0056238 A1
(43) Pub. Date: Mar. 2, 2017

(54) STIMULATION SYSTEM, DEVICE, AND METHOD FOR USE THEREOF

(71) Applicant: PHYSIOCUE, INC., SUNNYVALE, CA (US)

(72) Inventors: Seon YI, San Jose, CA (US); Jose BOLANOS, Orinda, CA (US); Leo SOONG, Walnut Creek, CA (US)

(21) Appl. No.: 15/256,342

(22) Filed: Sep. 2, 2016

Related U.S. Application Data
(63) Continuation of application No. PCT/US2015/018324, filed on Mar. 2, 2015.
(60) Provisional application No. 61/947,136, filed on Mar. 3, 2014.

(51) Int. Cl. A61F 7/00; A61N 7/00

(52) U.S. Cl. CPC A61F 7/007; A61N 7/00; A61F 2007/0009; A61F 2007/0075; A61F 2007/0093; A61F 2007/0094; A61F 2007/0096

(57) ABSTRACT

Provided herein is a stimulation device, system and method for therapeutic treatment of a condition or disease. In various aspects, the device may include one or more modules for low temperature, vibration, pressure, or ultrasound stimulation, operably connected to a contact tip; a data logging module; and a wireless communication module. Also provided is a system including such device and a remote server. Additionally, a method of treatment using such device or system is provided.

### CROSS REFERENCE TO RELATED APPLICATIONS

This application is a continuation of PCT patent application No. PCT/US2015/018324, with an international filing date of Mar. 2, 2015, published as WO2015/134394, entitled “STIMULATION SYSTEM, DEVICE, SYSTEM AND METHOD” which claims benefit/priority to U.S. Provisional Application No. 61/947,136, filed Mar. 3, 2014, and entitled “STIMULATION DEVICES AND METHODS OF USE THEREOF”, all of which is incorporated herein by reference in its entirety.

### INTRODUCTION

Various diseases, disorders, or conditions affect a significant proportion of the population. For example, essential hypertension is a global chronic disease problem. Essential hypertension can result from various factors including obesity, diabetes, smoking, lack of exercise, sodium intake, chronic anxiety, chronic stress, and aging. All of these contributing factors can cause hardening of the arteries, increased blood pressure, or excessive work load on the heart or vessels in the brain producing a substantially increased risk for heart attacks and strokes.

The autonomous nervous system including baroreceptors in the carotid and aortic arteries when in a normal healthy condition, regulate blood pressure by activating the parasympathetic nervous system by way of the vagus nerve. The autonomic nervous system has two main arms that counter-balance each other: the sympathetic nervous system is activated by stress and danger, and increases blood pressure and heart rate; whereas, the parasympathetic nervous system is the opposite, the relaxation or healing phase which brings down blood pressure and heart rate.

When hypertension is present, the sympathetic arm is over-activated and dominant aver the parasympathetic. The realities of our advanced society provide excessive stress and stimulus, aver prolonged periods of time, causing a systematic increase in blood pressure. This is a major concern as a chronic illness with serious potential outcomes.

Medical treatments for hypertension have traditionally been oral agents that affect different parts of the vascular, autonomic, and renal systems. Many oral agents, however, result in unwanted side effects. Further, the cast of such agents may be prohibitive. Holistic treatments have used relaxation stimulus, exercise, and biofeedback to attempt to lower blood pressure with poor effect.

Various devices for treatment of hypertension are also available. One such device of the related art is described in U.S. Pat. No. 7,713,295. Such devices typically rely on application of a single-temperature contact tip to a specific application site in an attempt to bring about the desired results. Some users may find the device somewhat unwieldy, e.g., struggle to correctly locate the device for the duration of treatment.

Further, current indications in the field of treatment suggest that a range of temperatures and a variety of treatment modalities may result in increasingly—beneficial health outcomes when used alone or in combination with one another.

What is needed, then, is an easy-to-use and cost-effective device, system and method that provides the functionality necessary to enhance or improve beneficial outcomes of stimulus treatment, with few or no adverse side effects.

### SUMMARY

Various aspects include a device comprising a case; a contact tip operably connected with the case; a low temperature stimulation module; and at least one of a vibration stimulation module, a pressure stimulation module, or an ultrasound stimulation module; wherein, the contact tip is suitable for contact with an external tissue of a subject; the contact tip is operably connected to the low temperature stimulation module such that the contact tip provides low temperature stimulation; and the contact tip is operably connected to the vibration stimulation module, the pressure stimulation module, or the ultrasound stimulation module, such that the contact tip provides vibration stimulation, pressure stimulation, or ultrasound stimulation, respectively.

Various aspects include a system comprising: a device comprising:

a case; a contact tip operably connected with the case; a low temperature stimulation module; at least one of a vibration stimulation module, a pressure stimulation module, or an ultrasound stimulation module; a data logging module; and a wireless communication module wherein, the contact tip is suitable for contact with an external tissue of a subject; the contact tip is operably connected to the low temperature stimulation module such that the contact tip provides low temperature stimulation; the contact tip is operably connected to the vibration stimulation module, the pressure stimulation module, or the ultrasound stimulation module, such that the contact tip provides vibration stimulation, pressure stimulation, or ultrasound stimulation, respectively; at least one of the low temperature stimulation module, the vibration stimulation module, the pressure stimulation module, or the ultrasound stimulation module is operably connected to the data logging module; and the data logging module is operably connected to the wireless communication module; and a remote data server, wherein the wireless communication module of the device communicates directly or indirectly with the remote data server.

Various aspects include a method of external stimulation for achieving a beneficial health outcome, the method consisting of the steps of: applying a contact tip of a device to an external tissue of a subject; and administering at least one of low temperature stimulation, vibration stimulation, pressure stimulation, or ultrasound stimulation to the external tissue of the subject for a period of time, wherein the device comprises the contact tip operably connected with a case; a low temperature stimulation module; and at least one of a vibration stimulation module, a pressure stimulation module, or an ultrasound stimulation module; wherein, the contact tip is suitable for contact with an external tissue of a subject; the contact tip is operably connected to the low temperature stimulation module such that the contact tip provides low temperature stimulation; and the contact tip is operably connected to the vibration stimulation module, the pressure stimulation module, or the ultrasound stimulation module, such that the contact tip provides vibration stimulation, pressure stimulation, or ultrasound stimulation, respectively.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a drawing of a device of the prior art.

FIG. 2 is a schematic diagram of a therapeutic treatment device, according to the present invention.

FIG. 3 is a schematic of the low temperature module of FIG. 2, according to the present invention.

FIG. 4 is a schematic of an additional functionality module, according to the present invention.

FIGS. 5 and 6 show a collar operably associated with the therapeutic treatment device of FIG. 2, according to the present invention.

FIG. 7 is a schematic of a system including the therapeutic treatment device of FIG. 2 and a remote data server, according to the present invention.

FIG. 8 is a flow diagram of an aspect of a method of external stimulation for achieving a beneficial health outcome, according to the present invention.

FIG. 9 shows one embodiment of the device of FIG. 2, according to the present invention.

FIG. 10 shows an exemplary cooperative environment associated with the device of FIG. 2, according to the present invention.

FIG. 11 shows an exemplary method associated with a logger, according to the present invention.

FIGS. 12 and 13, show a subject and corresponding treatment sites, according to the present invention.

FIG. 14 depicts the skull of a subject and an exemplary application site on the skull, according to the present invention.

FIGS. 15-17 are flow diagrams of a method of treating a headache, according to the present invention.

### DETAILED DESCRIPTION

Generally, the present disclosure provides a device, system, and method for treatment of a disease, disorder, or condition, such as hypertension, by stimulating a spot on the body of a subject, e.g., a carotid sinus, through one or more therapeutic modalities, e.g., cold, pressure, vibration, or ultrasound, with reduced or no side effects compared to conventional pharmaceutical administration and with longer lasting effects compared to conventional non-pharmaceutical techniques.

The device of the present disclosure provides, inter alia, optional features such as multiple contact tips, customizable temperature profiles including monitoring and automatic adjustment, wireless Bluetooth connectivity, data logging, and integration into a system that includes a remote data cloud server.

By providing data logging into a secure cloud storage platform, large data volumes can be stored with secure personal data. Using the saved data, a medical or health professional and family members, e.g. a doctor, nurse, data analyst, statistician, or physician's assistant and family members, user, or subject can perform more refined analyses and enhance treatment. A wireless Bluetooth connection can be used between an embedded data logging module and the contact module for the subject to use the handheld device and other operational components, e.g., smartphones, other mobile devices, and computers, etc., of the system in a comfortable position. Furthermore, one data logging module can operate several contact modules. A remote data server, e.g., a web-based server application, etc., makes it possible for medical or health professional, e.g. doctor, nurse or physician's assistant, a hospital or medical organization, users, or subjects to systematically manage and analyze a subject's collected data, e.g., blood pressure data, location position.

This provides a more efficient, usable, and efficacious system, method, device as further described.

Device

The present disclosure provides a device useful for a disorder, e.g., hypertension treatment, etc., via stimulating a point on the body of a subject, e.g., carotid sinus, with pressure, low temperature, vibration, ultrasound, or a combination thereof. In some embodiments, the device includes a pressure mechanism, a cooling mechanism, a vibration mechanism, or an ultrasound mechanism providing for stimulation, e.g., pressure, cold, vibration, or ultrasound stimulation, which can be repeated, etc., of a point for carotid body stimulation.

A device described herein can include a contact module with low temperature, vibration, pressure, or ultrasound stimulus function; a wireless communication module, e.g., short-wave radio, Bluetooth, near-field communications, etc.; application software for the operation of an embedded mobile port for data logger, and for each device or module; a UART expansion board to connect the embedded data logging module to each device; or a remote data cloud server application that can provide data and be viewed by medical or health professionals, family, users, or subjects, etc.

Various device embodiments can include a temperature sensor for sensing a cooling temperature of a contact tip in contact with a point on the body of subjects (carotid sinus) with optional automatic temperature adjustment to match a preset or selected temperature of the device. Also provided in the present disclosure the device may include a heat sink, cooling fan, and thermoelectric Peltier module for lowering the temperature of the tip of the device.

In one embodiment, a cooling device is contacted with a subject's carotid artery baroreceptor reflex location, where the cooling device stimulates the point of contact with a low temperature, e.g., about 0° C.±5° C., aver a period of time, e.g., approximately 5 to 10 minutes, etc. A contact tip in some embodiments of the device can maintain a temperature of, for example, about 0° C.±5° C. when not in contact with a point on the body of a subject.

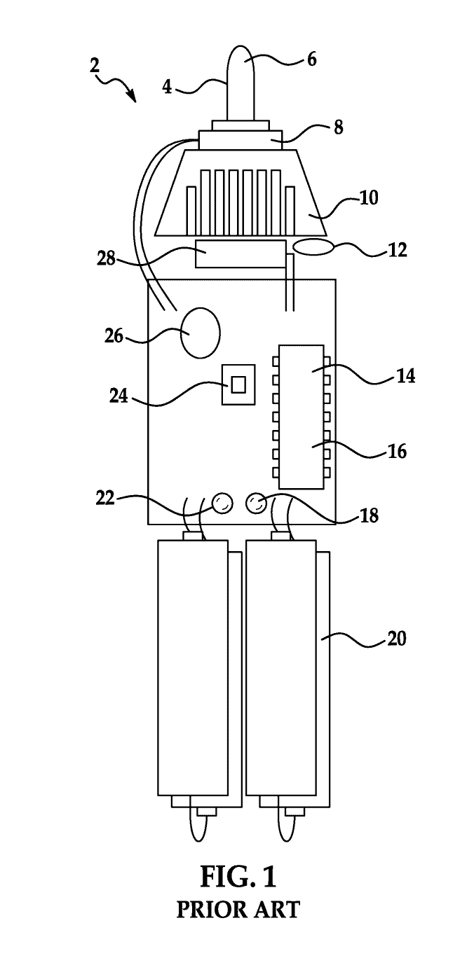

A skilled artisan will recognize that the device disclosed herein can include one or more features of the prior art treatment device 2 shown in FIG. 1 of the prior art. The prior art treatment device has a contacting tip 4, a magnetic portion 6, a thermoelectric module 8, a heat sink 10, a vibration motor 12, a PCB board 14, a one-chip microprocessor 16, an action lamp 18, a battery 20 a battery lamp 22, a power switch 24, a buzzer 26, and a cooling fan 28. The prior art treatment device 2, for example, is described in U.S. Pat. No. 7,713,295, the disclosure of which is herein incorporated by reference in its entirety.

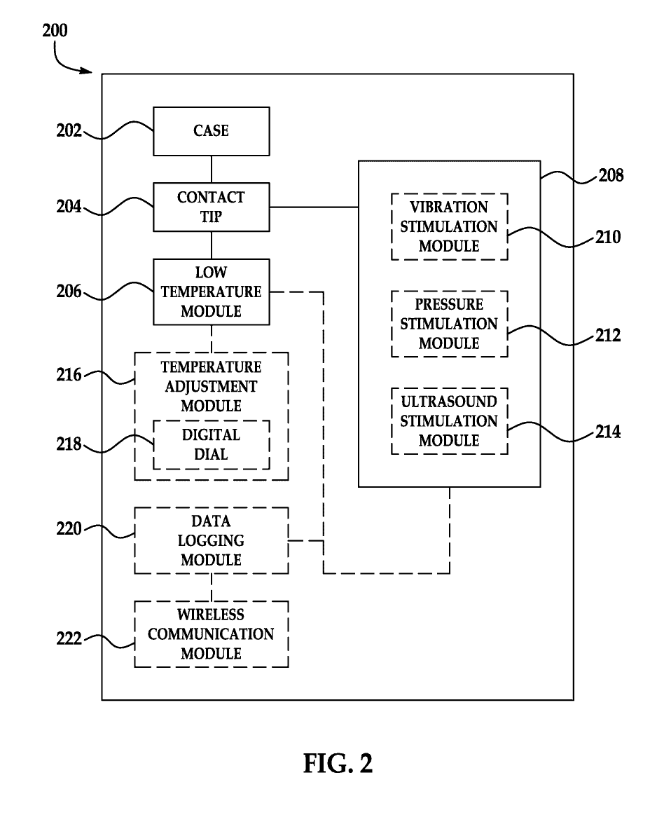

With reference now to FIG. 2, there is shown a schematic diagram of a device 200 comprising a case 202; a contact tip 204 operably connected with the case 202; a low temperature stimulation module 206; and an additional module 208 comprising at least one of a vibration stimulation module 210, a pressure stimulation module 212, and an ultrasound stimulation module 214.

The contact tip 204 is suitable for contact with an external tissue of a subject; the contact tip 204 is operably connected to the low temperature stimulation 206 module such that the contact tip 204 provides low temperature stimulation; and the contact tip 204 is operably connected to the vibration stimulation module 210 the pressure stimulation module 212, or the ultrasound stimulation module 214, such that the contact tip provides vibration stimulation, pressure stimulation, or ultrasound stimulation, respectively. One skilled in the art will appreciate that the foregoing modules may be implemented in a variety of ways so long as each is capable of performing or providing its respective functionality.

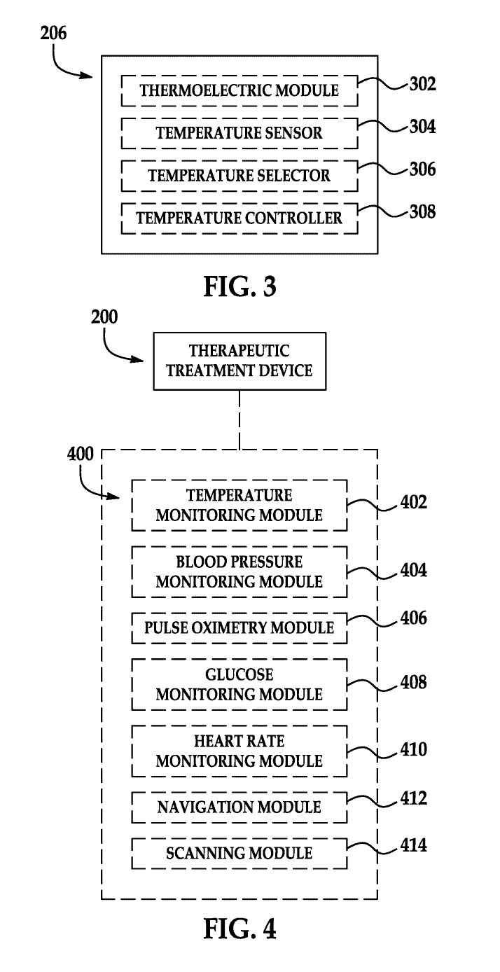

With continuing reference to FIG. 2 and with reference to FIG. 3, where there is shown a detailed schematic of the low temperature module of FIG. 2, one skilled in the art will note that the low temperature stimulation module 206 comprises one or more of a thermoelectric module 302, a temperature sensor 304, a temperature selector 306, or a temperature controller 308.

As further shown in FIG. 2 and in various aspects, the therapeutic treatment device 200 may further include one or more of a temperature adjustment module 216, which itself may include, for example, a digital dial 218, a data logging module 220, or a wireless communication module 222.

With reference to FIG. 4, there is shown a schematic of an additional functionality module 400 operably associated with the therapeutic device 200. The addition functionality module 400 comprises one or more of a temperature monitoring module 402, a blood pressure monitoring module 404, a pulse oximetry monitoring module 406, a glucose monitoring module 408, a heart rate monitoring module 410, a navigation module 412, and a scanning module 414.

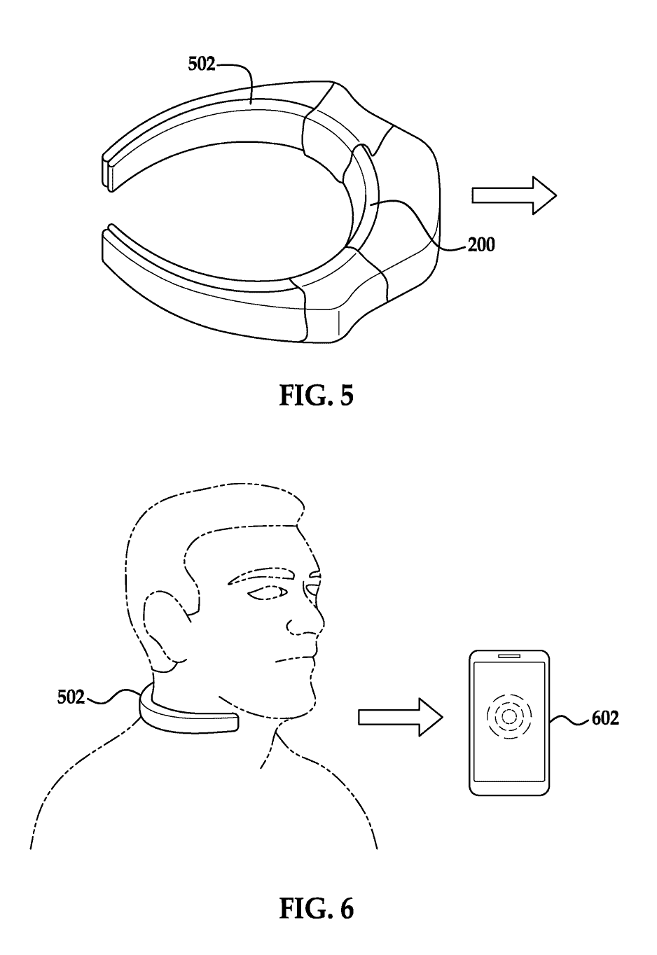

In various aspects and with reference to FIGS. 5 and 6, there are shown a collar 502 that can be operably associated with therapeutic treatment device 200. The collar 502 may be removably connectable to the case (202 of FIG. 2), for example, wherein the collar 502 provides for at least one of positioning the device on or near the tissue of the subject and positioning the device in electrical communication with a battery charging unit (not shown). For example, the collar 502 and device 200 may removably couple in such a manner, e.g., snap-in, clip, etc., that when the two engage and the collar 502 is positioned on the subject's neck, the contact tip 204 is advantageously brought into contact with a desired application site on the neck. In another example, the collar may be worn in a manner similar to a headband wherein when engaged with the device 200, the contact tip 204 is brought into advantageous contact with a desired application site on the skull. One skilled in the art will recognize that other manners and location of wear are possible.

Further, in various aspects and as depicted in FIG. 6, the collar 502 and/or the therapeutic treatment device 200 may be in communication with another device, e.g., a mobile phone 602.

For illustrative purposes, some aspects of the device 200 can include a heat sink, e.g., a metal heat sink, with good thermal conductivity, a conical cooling pin having a number of miniaturized vibration motors arranged at regular intervals to radiate or remove heat to the outside efficiently, a coupling slit formed in the front end thereof to couple a cap, or through holes formed in the side surface to draw out electric wires.

The contact tip 204 can protrude from an upper end portion of the case 202 or left or right side of a collar 502 device so as to be in contact with a point on the body of the subject, e.g., carotid sinus, and a thermoelectric Peltier module or module similar in functionality can be disposed on a lower end of the contact tip 204 to cool down the temperature of the contact tip to the temperature set by the user.

In some aspects, a thermoelectric Peltier module can be mounted on the heat sink where a heat generating surface of the thermoelectric Peltier module is directed to the heat sink, or a cooling surface of the thermoelectric Peltier module can have a contact tip, which can have a hemispheric magnet mounted at the top of the thermoelectric Peltier module. The thermocouple of the thermoelectric module can be drawn out of the lower end of the heat sink through the holes formed in the side surface of the heat sink. The contact tip can have an optimal volume to transmit cooling temperature, which can be set by a user or predetermined by a manufacturer, to a contact point on the body of a subject, sometimes referred to herein as an “application site.” A cooling fan can be mounted on or near the heat sink, e.g., at the lower-end portion of the heat sink, to effectively remove heat emitted from the heat generating surface of the thermoelectric Peltier module, thereby maximizing a cooling efficiency of the thermoelectric module.

In various aspects, the collar 502 and/device 200 is capable of providing pressure, cold, or vibration stimulation to a point on the body of a subject that can additionally feature one or more of the following features: wireless integration (e.g., Bluetooth, Ants, WIFI, based interface); temperature control adjustment, control or variability; interchangeable tips; concurrent or serial monitoring of parameters including, but not limited to, blood pressure, glucose, heart rate, pulse oximetry, movement, electrical conduction, electrical sensing, location positioning assistance, rechargeable batteries or a combination thereof.

The contact tip 204 that may be used, for example, to contact a patient's carotid sinus (Carotid Artery Baroreceptor Reflex) portion, may protrude upwardly from the upper end portion of the case.

Contact Tip

Aspects disclosed herein can include one or more contact tip. A contact tip can contact a spot on the body of a subject so as to deliver one or more of temperature stimulation, pressure stimulation, vibration, or ultrasound stimulation. A contact tip can contact a spot on the body of a subject so as to allow function of a monitoring module, such as temperature, pressure, blood pressure, oximetry, navigation, or scanning.

A contact tip can be operably connected to other modules described herein, including but not limited to the low temperature stimulation module, the vibration stimulation module, a pressure stimulation module, an ultrasound module, a navigation module, a data logging module, a wireless module, or a monitoring module described herein (e.g., blood pressure, glucose, heart rate).

A device can include contact tip variations. For example, a device can include interchangeable contact tips. As another example, a device can include different sized interchangeable tips that accommodate different sized subjects or for treating a smaller or larger part of the subject. A device can include one or more thicker tips.

A device can include one or more contact tips that can provide electrical stimulation to a spot on the body of the subject. A device can include one or more contact tips that can provide a vibrating type of action. A device can include one or more contact tips that can aid with navigation or locating the proper treatment location, e.g., a sensor for a pulse or electrical nerve conduction location. A device can include one or more contact tips that can provide various sensing capabilities, such as blood pressure, glucose monitoring, heart rate, or other monitored parameters as described herein. A contact tip can be connected to, detachably connected to, or integrated into a treatment tip. For example a contact tip can be alongside of or embedded in a treatment tip.

Temperature Stimulation

A device disclosed herein can include a temperature function. For example, a device disclosed herein can include a temperature module that measures, monitors, logs, or transmits a temperature stimulus, e.g., cold stimulus, to a point of the body of a subject.

A temperature stimulation module can be operably connected to one or more other modules described herein.

In some aspects, the device can provide low temperature stimulation via a contact tip point. The low temperature stimulation can be about 0° C.±5° C. For example, temperature stimulation can be about −4° C. to about 4° C. As another example, the low temperature stimulation can be about −3° C., about −2° C., about −2° C., about −1° C., about −1° C., about 0° C., about 0° C., about −1° C., about 1° C., about 2° C., about 2° C., about 3° C., about 3° C., about 4° C., about 4° C., about 5° C. Recitation of each of these discrete values is understood to include ranges between each value. A contact tip can be held at any of the above recited temperatures when in contact with a point on the body of a subject. The contact tip can be held at a lower temperature when not in contact with a subject. A contact tip can be held at any of the above recited temperatures when not in contact with a point on the body of a subject. For example, a contact tip can be about −5° C. to about 5° C. when not in contact with a point on the body of a subject.

A temperature stimulation can provide additive or synergistic therapeutic effects when combined with a vibration stimulus or pressure stimulus.

Thermoelectric Module

In various aspects, the thermoelectric module is mounted at an upper end portion of a case or the left or right side portion of the contact tip to cool the contact tip to a cooling temperature set by the user according to input current. The thermoelectric module has a cooling surface formed on the upper surface thereof, which is in contact with the contact tip, and a heat generating surface formed on the lower surface thereof, which is in contact with a heat sink One or more features described herein can be included in these embodiments.

In various aspects, a heat sink is mounted on the lower portion of the thermoelectric module, and has a cooling fan mounted at the lower end thereof for discharging heat emitted from the heat sink to the outside so as to improve a cooling efficiency of the thermoelectric module. Therefore, when the thermoelectric module is operated, the cooling fan can also operate thereby quickly and efficiently removing heat from the heat sink in contact with the thermoelectric module using forced convection, so that the thermoelectric module has improved cooling efficiency. One or more features described herein can be included in these embodiments.

After the therapy operation is started, when the operator/user contacts the cooled contact tip onto the patient's carotid sinus baroreceptor reflex point for a period of time, e.g., about 3 to about 10 minutes, a condition of elevated blood pressure can be reduced effectively.

As a result of stimulating the patient's carotid sinus portion with, for example, pressure, cold, or vibration, with a device disclosed herein, the numerical value of blood pressure can be lowered. For example, a study of 60 patients, who had blood pressure defined as stage 1 and 2, were stimulated at a low temperature of 0° C.±5° C. for about 5 minutes daily for 69 days, and the blood pressure of the hypertensive patients was reduced by 10-30 mmHg in 87% of the patients and considered as moderate to significant by the researchers. It was confirmed that the stimulation was harmless and painless to the patients and that the effect was mostly local stimulation to the carotid sinus area for that short duration of time.

Control Module

Various aspects can include a controlling device for maintaining the cooling temperature of the contact tip set by a user.

In various aspects and with reference to, there is shown one embodiment of device 200 which includes, for example, a contact module, e.g., thermoelectric module (sumer electric module 902), a radiator 904 or fan 906, temperature sensor 908, vibrator 910 etc., and a control part, e.g., micro controller 912. A control module can be driven by a source of electrical power, e.g., rechargeable battery. A control module can also receive data from the data logging module for transmission to a predetermined destination(s), e.g., the cloud platform for archiving treatment blood pressure readings and before and after blood pressure data.

A controlling device can include a key input part, e.g., a switch, etc., starting or terminating operation, an LED for displaying the present status, or a buzzer for informing start and termination of operation to the user. A device described herein can include one or more features of the controlling device.

In some aspects, a user can supply or trigger a power source by, for example, manipulating the key input part, and a buzzer can inform the user of start of 20 operation by a continuous sound or an LED light can indicate application of the power source. The controlling device can control the strength of electric current supplied to the thermoelectric Peltier module according to a supplied control signal, and the thermoelectric Peltier module can cool the contact tip according to the electric current supplied from the controlling device.

In some aspects, a temperature sensor mounted on the contact tip can sense the present cooling temperature of the contact tip or transmit it to a temperature controller. The temperature controller can compare present cooling temperature sensed by the temperature sensor with the temperature of the contact tip set by the user. After that, the temperature controller can output a control signal, which can be modulated in pulse width corresponding to the temperature difference between the set temperature and the present cooling temperature, to a current controller. The current controller can control the strength of electric current supplied to the thermoelectric Peltier module according to, for example, the pulse width modulated control signal inputted from the temperature controller, so that the cooling temperature of the contact tip can be maintained at the cooling temperature set by the user.

Furthermore, when the thermoelectric module is operated, the cooling fan can also be operated so as to emit heat from the thermoelectric module and improve cooling efficiency. Cooling efficiency can allow, for example, the contact tip to reach a desired cooling temperature faster.

A control module can be integrated in the device including the contact module or be contained in a separate device.

Vibration Stimulation

A device disclosed herein can include a vibration function. For example, a device disclosed herein can include a vibration module that measures, monitors, logs, or transmits a vibration stimulus to a point of the body of a subject.

A vibration stimulation module can be operably connected to one or more other modules described herein.

Vibration stimulation can provide additive or synergistic therapeutic effects when combined with a temperature stimulus or pressure stimulus.

Pressure Stimulation

A device disclosed herein can include a pressure function. For example, a device disclosed herein can include a pressure module that measures, monitors, logs, or transmits a pressure stimulus to a point of the body of a subject. A pressure module can also measure, transmit, or log the duration of time for which there is pressure stimulus applied to a point of the body of a subject. For example, duration time of a single treatment or multiple sequential or non-sequential treatments can be determined, transmitted or logged. A pressure measurement function can also provide for feedback the user with respect to applied pressure or optimal pressure.

A pressure stimulation module can be operably connected to one or more other modules described herein.

Pressure stimulation can provide additive or synergistic therapeutic effects when combined with a temperature stimulus or vibration stimulus.

Pressure stimulation can be applied in varying degrees of force. For example, a tip can be controllable or controlled via a spring activated mechanism or solenoid valve. Similarly, the pressure can be varied by use of an air pressure mechanism.

Ultrasound Stimulation.

A device disclosed herein can include an ultrasound function. For example, a device disclosed herein can include an ultrasound module that can stimulate a spot on the body of the subject. A device can include an ultrasound diagnostic imaging module. A device can include an ultrasound therapeutic module. A device can include a mechanism for emitting ultrasonic, i.e., ultrasound, wavelength ranges capable of stimulating or treating a location as desired, e.g., the carotid artery. An ultrasound module can be turned on or off, along with or independent of other modules of the device. Accordingly, focused ultrasonic energy can delivered to a spot on the body of the subject, e.g., a soft tissue area, desired to be treated. The ultrasound module can provide a non-ablative energy. A gel can be used in conjunction with a device featuring an ultrasound module, which can assist transmission of the ultrasonic waves or reduce friction.

An ultrasound stimulation module can be operably connected to one or more other modules described herein.

An ultrasound module can deliver pulses of ultrasonic energy. A pulse duration, e.g., the time during which the energy is activated, can be about 2 ms, i.e., 2 thousandths of a second. An off period can be variable or fixed. Exemplary pulse ratios include from about 1:1 to about 1:4, e.g., about 1:2 or about 1:3. In 1:1 mode, an ultrasound module can output for about 2 ms followed by an about 2 ms rest. In 1:4 mode, an about 2 ms output can be followed by an about 8 ms rest period. The above diagram illustrates the effect of varying the pulse ratio.

Additional exemplary pulse ratios include about 1:5, about 1:6, about 1:7, about 1:8, about 1:9, or more. A pulsing algorithm can be expressed in percentage rather than a ratio, e.g., 1:1=50% 1:4=20%, etc. The proportion of time that the device is ON compared with OFF can be a relevant factor in dosage calculations.

Temperature Monitoring

Various aspects disclosed herein can include a temperature measurement or adjustment function. For example, a device disclosed herein can include a temperature measurement module that measures, monitors, logs, or transmits a temperature of the contact tip of the device when in contact or when not in contact stimulus with a point of the body of a subject. As another example, a device disclosed herein can include a temperature monitoring module that can measure the temperature of a contact tip of the device when in contact with a spot on the body of a subject or not in contact with a spot on the body of a subject. As another example, a device disclosed herein can include a temperature adjustment module that adjusts temperature of the contact tip of the device when in contact or when not in contact stimulus with a point of the body of a subject.

A temperature monitoring module can be operably connected to one or more other modules described herein.

A temperature sensor can be built into the device or a module thereof. For example, a temperature sensor can be built into an interchangeable tip.

A temperature sensor can measure a range of variables during use of the device. For example, a temperature sensor can measure temperature of a contact tip over time, minimum temperature reached, maximum temperature reached, or average temperature maintained.

Temperature adjustment, e.g., manual or automatic, can provide for systematic or efficient management of treatment via optimal conditions, e.g., temperature of carotid artery baroreceptor reflex contact tip, time/hours of use, etc., or can allow a subject to adjust parameters for convenience, comfort, safety, or efficiency of treatment.

A device can include a digital or other means to modify or adjust the temperature of one or more contact tips, i.e., a temperature adjustment module. For example, a device can include a digital dial. A device can transmit data parameters, e.g., temperature, to a mobile device, such as but not limited to a mobile phone, tablet, laptop, “smart” type watch, headband, wrist band or wearable “health and wellness” type device. A device can adjust temperature via a remote connection, such as IR hand-held device, WIFI, Mobile, SaaS, Cloud, fiber optic, or Ethernet connection.

A temperature measurement module can be integrated in the device including the contact module or be contained in a separate device.

A temperature stimulation can provide additive or synergistic therapeutic effects when combined with a vibration stimulus or pressure stimulus.

Temperature Control Circuit

A device described herein can include a circuit, for maintaining the cooling temperature of the contact tip set by a user. In some embodiments, the temperature sensor can include a thermistor or resistances R5 or R6, which have resistance values varied according to a temperature change of the surroundings. When the temperature of the contact tip is changed, the temperature controller can sense the present cooling temperature of the contact tip while the resistance value of the thermistor is varied. The key input part can include a number of switches (e.g., SW1-SW4) or resistances (e.g., R1-R4) for allowing the user to input a desired setting temperature. The device can include a buzzer, or other audio or sensory alert.

Blood Pressure Monitoring

A device disclosed herein can include a blood pressure monitoring function. For example, a device disclosed herein can include a blood pressure monitoring module that can measure the blood pressure of subject before, during, and/or after use of the device.

A blood pressure monitoring module can be operably connected to one or more other modules described herein.

A blood pressure monitoring module can generally capture blood pressure readings of a subject. A blood pressure monitoring module can record blood pressure readings. A blood pressure monitoring module can transmit blood pressure readings via the Cloud, Bluetooth, ANTS, RFID, WIFI, Hand-held device, such as a mobile phone or tablet, a desktop or laptop computer, a wrist watch, a wearable “health and wellness” type of device, an arm band, a ring, an eye glass, contact lens, or Mobile and SaaS.

Blood pressure can be measured by any suitable device, including but not limited to a wrist, finger, or arm-based blood pressure monitoring cuff.

A blood pressure monitoring module can include, for example, an aneroid monitor or a digital monitor. A type of blood pressure measurement can be chosen by a subject or a health care provider. An aneroid monitor can have a gauge readable by looking at a pointer on a dial. A cuff can be placed on or around a wrist, finger, or arm of a subject and inflated automatically or manually, e.g., by squeezing a rubber bulb. A digital blood pressure monitor can have a manual or an automatic cuff. The blood pressure reading can be displayed on a small screen. The digital monitor can be a standalone unit, or it can be integrated into a device such as a mobile phone, tablet, laptop, watch, headband, contact lens, or eyeglasses.

A blood pressure monitor can be integrated into, for example, a mobile, tablet, watch, headband, ring, laptop, web, WIFI, cloud-based or other type of technology, or may be app based, such as a Blood Pressure Monitoring application.

Pulse Oximetry Monitoring

A device disclosed herein can include a pulse oximetry monitoring function. For example, a device disclosed herein can include a pulse oximetry monitoring module that can measure a subject's oxygen saturation before, during, or after use of the device.

An oximetry sensor can be built into the device or a module thereof. For example, an oximetry sensor can be built into an interchangeable tip.

Glucose Monitoring

A device disclosed herein can include a glucose monitoring function. For example, a device disclosed herein can include a glucose monitoring module that can measure a subject's blood glucose level before, during, or after use of the device.

A glucose monitoring module can be operably connected to one or more other modules described herein.

A glucose sensor can be built into the device or a module thereof. For example, a glucose sensor can be built into an interchangeable tip.

Heart Rate Monitoring

A device disclosed herein can include a heart rate monitoring function. For example, a device disclosed herein can include a heart rate monitoring module that can measure a subject's heart rate level before, during, or after use of the device.

A heart rate monitoring module can be operably connected to one or more other modules described herein.

A heart rate sensor can be built into the device or a module thereof. For example, a heart rate sensor can be built into an interchangeable tip.

Navigation Module

A device disclosed herein can include a navigation feature. For example, a device disclosed herein can include a navigation module.

A navigation module to assist in correct application of the device can be operably connected to one or more other modules described herein.

A navigation module can be integrated at or near the contact tip of the device.

A navigation module can be integrated into the device such that it is tethered, via a wireless connection, or direct connection such as a fiber optic, electrical wire, or other similar connection. Such tethered connection can be retractable or non-retractable. A tethered device can have one or more of an adhesive tip, a non-adhesive tip, a suction tip, or other securing mechanism sufficient to secure to a location for a period of time.

A navigation module can be provided as a separate device in system. For example, a navigation module can be an implant, which can remain at an implanted location, e.g., as chosen by a physician. An implanted navigation module can assist in locating a spot for treatment.

For example, a device disclosed herein can feature a navigation module with a pulsation sensor for identifying a pulse, such as a carotid artery pulse.

A navigation module can have a feedback mechanism, e.g., sound or light, such that a user can be alerted to one or more parameters, such as an optimal location for device placement to effect treatment.

As another example, a navigation module can include an electrical conductivity sensor. An electrical conductivity sensor integrated into the device or system can provide for identification of a spot on the body of a subject, such as the carotid artery baroreceptor reflex, or another nervous system based conduction location.

As another example, a navigation module can include a light, such as a blue or black light, that when turned on, can visibly “light up” a dot or any kind of marker that is placed on a subject's treatment area. The dot can be, for example, a temporary or permanent tattoo and can be used by a subject, user, nurse, physician assistant, doctor or the like to easily, quickly and more accurately find or re-find a point that requires treatment.

Wireless Module

In some embodiments, a device described herein can have a wireless communication module. Such wireless communication module can provide for remote medical service, e.g., tele-medical, also call RPM, Remote Patient Monitoring, by medical or health professionals. A device described herein can have internet, mobile or remote integratable technology. A wireless module can provide a commercially viable, active and implemented technology for RPM or telemedicine, which can be useful to, e.g., attending doctors, senior citizens, chronically ill patients, or office workers who are at home.

A wireless communication module can be operably connected to one or more other modules described herein.

A wireless communication module can receive or transmit data according to one or more of Cloud, Bluetooth, ANTS, RFID, WIFI, Hand-held device, such as a mobile phone or tablet, a wrist watch, a wearable “health and wellness” type of device, an arm band, a ring, eye glasses, a contact lens, or Mobile and SaaS.

Exemplary mobile wireless include Bluetooth enabled, RFID enabled, or synthesized and integrated into an RPM, Remote Patient Monitoring System, e.g., AT&T.

A wireless communication module can interface with internal device features or external separate devices. A device can include a wireless communication module that can interface with a blood pressure monitoring module, which can allow subject to measure the blood pressure before, during, or after treatment; vibrating stimulation module; a cold temperature stimulation module; or a data logger that can allow logging on to a remote data server or transmitting data thereto or therefrom.

Wireless communication, e.g., radiofrequency wireless communication) can eliminate a restriction of distance in a device. For example, one data logger can be used with multiple contact modules so as to maximize system efficiency.

A wireless communication module can include an industrial modem capable of transmitting data, e.g., contact tip temperature, contact pressure, blood pressure, time or duration of use, etc., to a remote server and the measurement instrument.

The device described herein can include: a contact module, a wireless communication module, a data logger, e.g., a GUI embedded data module, a blood pressure measurement module that can send data to the data logger through, e.g., Bluetooth interface, and an industrial modem capable of transmitting data to a remote cloud server.

A wireless communication module can include a Single-Mode BLE (Bluetooth Low Energy) Module Featuring smart BASIC. For example, a BL600 Series module (Laird Technologies) can provide for integration of single-mode Bluetooth Low Energy (BLE), or Bluetooth® Smart technology, to small, portable, power-conscious devices, e.g., devices powered by a small battery, such as AAA or coin cell. A programmable module that features such an event-driven smart BASIC programming interface can significantly simplify BLE module integration.

A wireless communication module including a BLE technology can be based on a Nordic Semiconductor nRF51822 chipset, and a BL600 module can provide exceptionally low power consumption with outstanding wireless range, and can fit within a compact footprint, e.g., 19 mm×12.5 mm. A wireless communication module can include hardware or firmware necessary or useful to support development of a BLE application, including but not limited to: optimized BLE radio hardware; UART, SPI, 12C, ADC, or GPIO interfaces, a complete embedded BLE software stack; a GATT-based BLE profile that can include Blood Pressure, Heart Rate, Health Thermometer, Find Me or Proximity; or Custom Services supporting one or more application needs.

Smart BASIC can make a wireless communication module unique, in that they are an event-driven programming language that enables standalone operation of a module whereby sensors can be attached via any of the interfaces without the need for an external processor. A simple smart BASIC application can encapsulate a complete end-to-end process of reading, writing, or processing of sensor data and then can use BLE to transfer such to a Bluetooth Smart device, e.g., a smartphone, tablet, gateway, or computer.

Various wireless communication modules can have FCC modular, IC, MIC, or CE approval. Various wireless communication modules can include use of a BL600 module that is fully qualified as a Bluetooth End Products, which can allow integration of a module in a device without requiring further Bluetooth Qualification.

A wireless communication module can include a wideband CMOS RF chip (Lime Microsystems), which integrates 2×2 MIMO functionality and supports all or most all cellular standards or frequencies, including 2G, 3G or 4G/LTE or TDD/FDD variants among other standards such as Wi-Fi. The zero IF transceiver has been taped out in 65 nm CMOS and can run from 50 MHz to 3.8 GHz. A CMOS RF chip can aid in the development of M2M and Internet of Things (IoT) devices. In some embodiments, a device described herein includes a SiGe BiCMOS (Lime Microsystems).

A device including an above described transceiver can support various markets and clinical indications. Furthermore, above described transceiver can reduce cost with high volume device manufacturing and allow for expandability of features. Integrating a transceiver described above can provide features such as DSP functions, a microcontroller, multiple 12 bit ADCs and DACs, LNAs, filters, PLLs, or mixers that can be accessed separately from the RF chain. These elements, alone or in combination, can provide cost-effective stand-alone parts and the open architecture, which can allow each function to be accessed or used separately. The DSP can enhance analogue gain or filtering with digital control and can be an important factor in reducing the overall power consumption. The LMS7002M can operate from a single supply rail of 1.8V with individual blocks capable of being powered down when not required for further power savings. Such features make it suitable for a wide range of battery and mains powered mobile communications devices, including a device described herein.

It is understood that some or all of the above described features and benefits can be in whole or in part provided through a wired interface to a separate computer or other device.

Data Logging Module

A device disclosed herein can include a data logging function. For example, a device disclosed herein can include data logging module that can stare, manipulate, or transfer data of one or more parameters discussed herein. A data logging module can be integrated in the device including the contact module.

A data logging module can provide an integrated management system for data collected or received by the device. For example, a data logging module can collect or receive data associated with temperature stimulus, vibration stimulus, pressure stimulus, blood pressure, treatment time, or treatment date. As another example, a data logging module can interface with a wireless communication module. Thus can be provided or improved an optimal condition of use of the device by, for example, a medical or health professional.

A data logging module can utilize X-scale based embedded and Mobile loaded data logger, which can be used in an embedded system such as HPC, PDA, smart phone, or mobile computer device.

A X-scale based embedded data logger can perform integrated management of a contact module, blood pressure monitor module, or wireless communication module. An exemplary device uses an X-scale based CPU (PXA255). An exemplary device features a GUI using a GTK library, which can increase user convenience.

GTK (gimp toolkit) is a library that builds a graphical user interface. A contact module and wireless communication module and a blood pressure measurement module can be connected to the data logging module. With reference to FIG. 10, there is shown an exemplary cooperative environment 1000 having an embedded data logger system 1002 operably associated with a database 1004. The embedded data logger system 1002 interfaces with a mux 1006, which in turn interfaces with an RF module 1008. The mux 1006 and I or RF module 1008 interface with various devices, e.g., an industrial modem 1010, a blood pressure measurement instrument 1012, a blood pressure depressor 1014 and its associated RF module, etc.

A data logger module can include an application program as a mobile touch screen based GUI, which can provide increased convenience for the user. For such embodiment, the embedded X-scale based embedded and GTK library for the GUI can port into an X-scale embedded system.

A device user, e.g., a hypertension patient, can save data, e.g., time of use, blood pressure before and after changes, etc., into an internal or external database. A device user can send data to a medical or health professional through a wireless communication module thereby providing for systematic, safe, and efficacious treatment.

A data logging module can transfer a subject's parameter data, e.g., hypertension reading, blood pressure, operating time, contact tip set temperature, contact tip operating temperature, before, during, or after use of the device to a remote server.

If “blood pressure monitor” on screen is selected, the screen can change to the blood pressure monitor screen and can show on the mobile device. The mode can show the performance measurement of the subject's blood pressure. In the blood pressure monitor mode, a subject can put on a cuff, then click the start button after which pressurization can start. Systolic, diastolic blood pressure or heart rate can be displayed on the screen. Once measurement is complete, the exit button can be clicked to move to the initial screen. If operation mode is selected on the screen, the screen can move to wireless monitoring and treatment-based operation mode. This mode can operate, e.g., a carotid artery baroreceptor reflex contact module.

To operate the device, a user can set the time of use or the contact temperature, then click the start button. The contact module and the blood pressure monitoring module can be operably connected. Parameters such as run time, temperature, or blood pressure can be displayed on the screen while the device is operating. After this operation, the user can click the exit button to go back to initial screen. A subject's data can be transferred when a user selects the transfer button.

Data Cloud Server (SAAS)

A device disclosed herein can interface with a data cloud server. For example, a device disclosed herein can interface through a wireless communication module with a data cloud server.

It can be difficult or impossible for a subject to efficiently analyze blood pressure trends without technological aid. A device described herein can, for example, transmit a subject's blood pressure data, or other data, to a medical or health professional remotely. A remote data server can accommodate or mediate such transmission.

A program can be implemented for reception or transmission of blood pressure data, or other data, from a subject. A program can explain a method for using the device, including feedback or instructions based on collected or transmitted data. A cloud based monitoring system can explain a method for using the device, including feedback or instructions based on collected or transmitted data, or collect, transmit, confirm, or update data (including personal data, such as medical history) related to the device, subject, or condition to be treated. A subject can interface with the device or the monitoring system through, for example, an internet connection.

A remote data server can be a server-based program used by a medical or health professional to tele-consult a subject who sent data (e.g., blood pressure data) by a wireless communication module or other means. A data cloud server system can be developed to send optimal conditions (e.g., proper temperature of contact tip, time of use, etc.) to a remote user. A mobile device can show the treatment history screen for the subject (e.g., a hypertension subject) received by the server program once sent through a wireless Bluetooth communication module from the database of the data logging module.

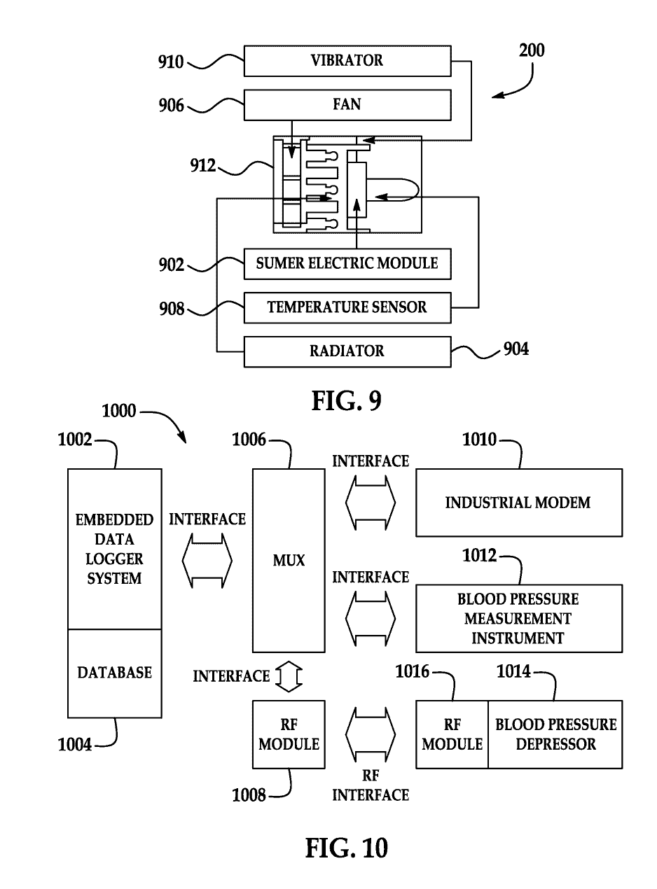

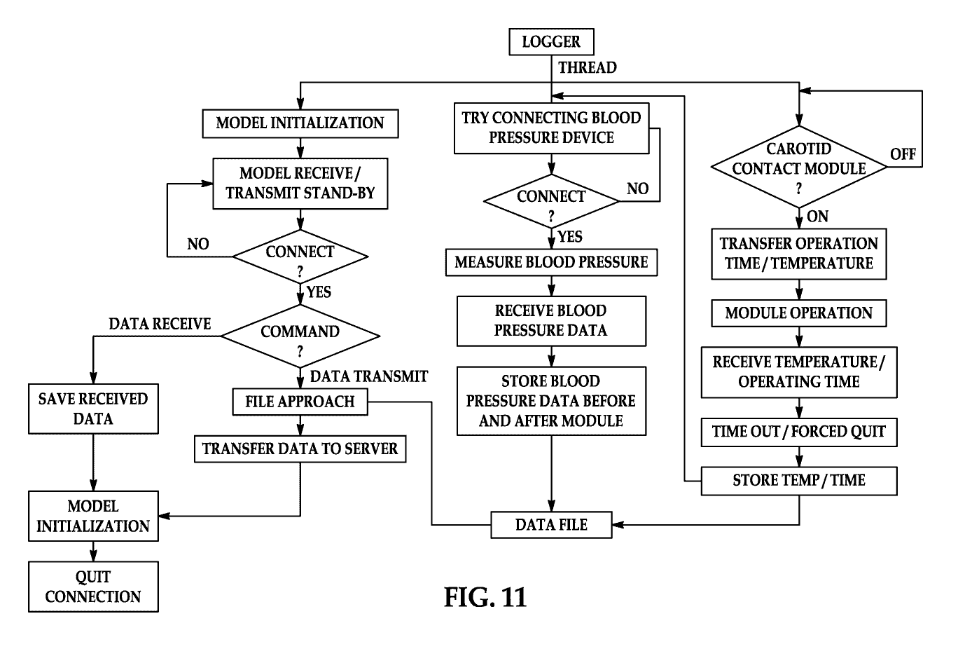

Operation of the system in accord with FIG. 9 can be as follows. A blood pressure monitor with the interface and contact module can be connected to the embedded SaaS and Mobile-based data logger to monitor, control, or adjust the temperature of the contact modules. Data parameters, e.g., measured blood pressure, temperature of a contact tip, time or date of use, before, during, or after using the device can be saved into a database and transmitted to the remote data server. Medical or health professionals or users that receive the data through the remote server program can send feedback or instructions, e.g., the optimal condition for contact tip temperature, the time of use, usage count based upon data the patient sent, etc., back to the subject.

Wearable Collar or Holder

A device can include a structure for holding the device or wearable collar device aver a spot on the body of the subject, e.g., a pulse point. In one embodiment, the device includes a collar integrated into or onto the device.

A device can be designed or shaped such that it can be placed into a holder that allows for easy and automatic re-charging of the rechargeable battery.

A device can feature a design and battery providing for recharging via induction. For example, a device can be placed onto an inductive charging pad so as to recharge the device. Charging can stop automatically when they are fully charged, so each battery is never overcharged. A compatible power clip, e.g., USB or mini USB, can be a component of the system.

A device can feature power monitoring. A device can feature an indicator, e.g., an audible beep, tone, or sound and visual indication such as a light meter, to indicate power remaining in the device.

### System

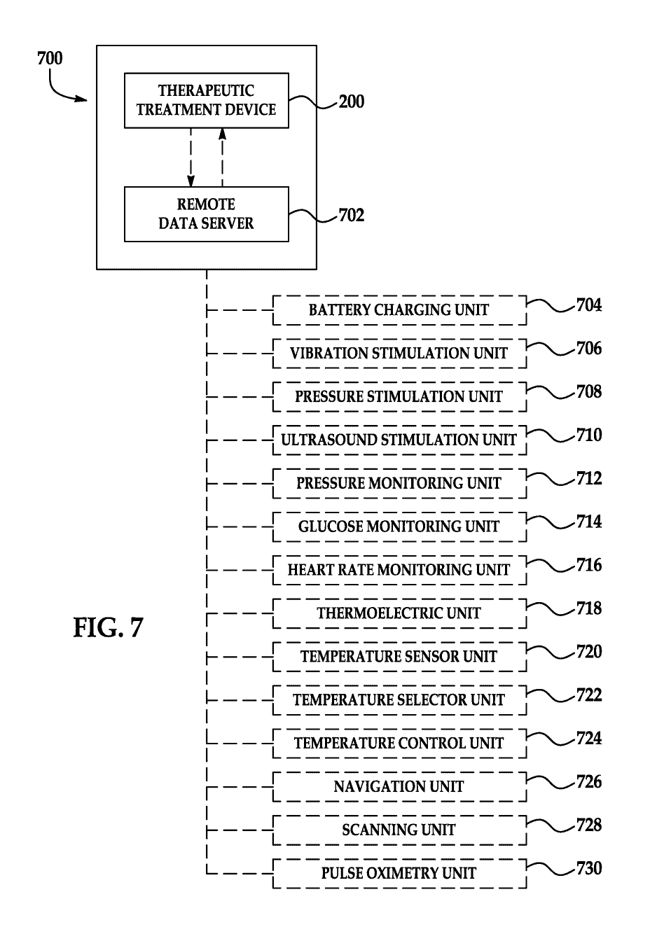

In various aspects and with continuing reference to the figures and with reference now to FIG. 7, there is shown a schematic of a system 700 which may include, for example, the therapeutic treatment device 200 and a remote data server 702. As previously discussed with respect to the therapeutic treatment device 200, various aspects of the system may include a case; a contact tip operably connected with the case; a low temperature stimulation module; at least one of a vibration stimulation module, a pressure stimulation module, or an ultrasound stimulation module; a data logging module; and a wireless communication module wherein, the contact tip is suitable for contact with an external tissue of a subject; the contact tip is operably connected to the low temperature stimulation module such that the contact tip provides low temperature stimulation; the contact tip is operably connected to at least one of the vibration stimulation module, the pressure stimulation module, and the ultrasound stimulation module, such that the contact tip provides vibration stimulation, pressure stimulation, and ultrasound stimulation, respectively; at least one of the low temperature stimulation module, the vibration stimulation module, the pressure stimulation module, or the ultrasound stimulation module is operably connected to the data logging module; and the data logging module is operably connected to the wireless communication module. The remote data server 702 may comprise any one or more devices, computers, components, etc., capable of sending and/or receiving data from the therapeutic treatment device 200. Examples of the remote data server include mobile phones and other handheld devices; websites, computers such as servers, desktops, and tablets, etc. The therapeutic treatment device 200 and the remote data server 702 may communication directly with one another or indirectly via, for example, mobile devices, tablets, computers, and the like. In one aspect, the remote data server 702 communicates directly or indirectly with the wireless communication module 222 of the therapeutic treatment device 200.

In various aspects, the system 700 may include one or more of the following: a battery charging unit 704, a vibration stimulation unit 706, a pressure stimulation unit 708, an ultrasound stimulation unit 710, a blood pressure monitoring unit 712, a glucose monitoring unit 714, a heart rate monitoring unit 716, a thermoelectric unit 718, a temperature sensor unit 720, a temperature selector unit 722, a temperature controller unit 724, a navigation unit 726, a scanning unit 728, and a pulse oximetry unit 730, any or all of which may be operably connected to the therapeutic treatment device 200 in a remote manner or in an integral manner.

### Method

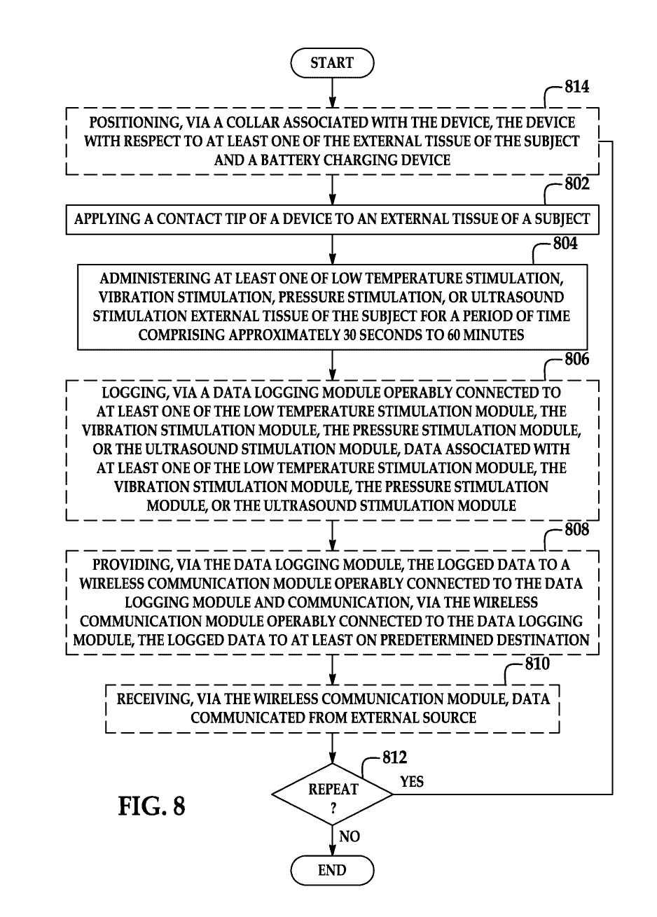

With continuing reference to the figures and with reference now to FIG. 8, there is shown a method 800 of external stimulation for achieving a beneficial health outcome. In various aspects, the method consisting of the steps of:

applying a contact tip of a device to an external tissue of a subject 802; and administering at least one of low temperature stimulation, vibration stimulation, pressure stimulation, or ultrasound stimulation to the external tissue of the subject for a period of time 804, e.g., where the period of time is approximately 30 seconds to 60 minutes, wherein the device comprises the contact tip operably connected with a case; a low temperature stimulation module; and

at least one of a vibration stimulation module, a pressure stimulation module, or an ultrasound stimulation module; wherein, the contact tip is suitable for contact with an external tissue of a subject; the contact tip is operably connected to the low temperature stimulation module such that the contact tip provides low temperature stimulation; and the contact tip is operably connected to the vibration stimulation module, the pressure stimulation module, or the ultrasound stimulation module, such that the contact tip provides vibration stimulation, pressure stimulation, or ultrasound stimulation, respectively.

In various aspects, the method 800 may further consist of one or more of the following steps: logging, via a data logging module operably connected to at least one of the low temperature stimulation module, the vibration stimulation module, the pressure stimulation module, or the ultrasound stimulation module, data associated with at least one of the low temperature stimulation module, the vibration stimulation module, the pressure stimulation module, or the ultrasound stimulation module 806; providing, via the data logging module, the logged data to a wireless communication module operably connected to the data logging module and communicating, via the wireless communication module operably connected to the data logging module, the logged data to at least one predetermined destination 808. In various aspects, the predetermined destination comprises at least one of a remote data server and a mobile device. In various aspects, the method 800 further consists of a step of receiving, via the wireless communication module, data communicated from an external source 810. The data may comprise, for example, at least one of low temperature stimulation data, vibration stimulation data, pressure stimulation data, ultrasound stimulation data, blood pressure data, glucose level data, heart rate data, spatial position with respect to the subject data, operation time data, tip-tissue contact time data, pulse oximetry data, or scanned information.

In various aspects, the method 800 further consists of the step of repeating the method (any step or combination of steps), for a predetermined number of times and at a predetermined interval 812. The method 800 may further consist of the step of positioning, via a collar associated with the device, the device with respect to at least one of the external tissue of the subject and a battery charging device 814. The step 814 may be, for example, an initial step in the method 800. In various aspects of the method 800, the beneficial health outcome comprises at least one of the following: lowered blood pressure, decreased anxiety, decreased respiratory rate, decreased muscle tone and relaxation, improved mental focus, and decreased inattention associated with attention deficit disorder.

Mechanism

While under no obligation to do so, and in no way limiting the scope of the present disclosure, a present understanding of one of various underlying physiological mechanism is provided.

The sympathetic nervous system is one of three major parts of the autonomic nervous system, along with the enteric and parasympathetic systems. General action of the sympathetic nervous system can mobilize the body's nervous system “fight-or-flight” response, but is also constantly active at a basic level to maintain homeostasis.

There are “baroreceptors” pressure sensors in positions of the body, such as the aortic arch or carotid bifurcation. Stimulation on the carotid sinus can cause an immediate lowering of blood pressure and heart rate. Long term blood pressure regulation is thought to be a component of the renal renin-angiotensin balance as a hormonal effect.

Studies described herein show that daily activation of the carotid sinus appears to cause a down-regulation of the sympathetic tone and an increase in the parasympathetic tone, thereby resetting a new balance in the autonomic system, a function that is hypothalamic in origin.

Arterial blood pressure is normally regulated within a narrow range, with a mean arterial pressure typically ranging from 85 to 100 mmHg in human adults. Tight control of this pressure ensures adequate blood flow to organs throughout the body. Such control is accomplished by negative feedback systems incorporating pressure sensors (i.e., barareceptors) that sense the arterial pressure Important arterial baroreceptors are located in the carotid sinus (at the bifurcation of external and internal carotids) and in the aortic arch. These receptors respond to stretching of the arterial wall so that if arterial pressure suddenly rises, the walls of these vessels can expand, which stimulates the firing of these receptors. If arterial blood pressure suddenly falls, decreased stretch of the arterial walls can lead to a decrease in receptor firing.

The carotid sinus baroreceptors are innervated by the sinus nerve of Herring, which is a branch of the glossopharyngeal nerve (IX cranial nerve). The glossopharyngeal nerve synapses in the nucleus tractus solitarius (NTS) are located in the medulla of the brainstem. The aortic arch baroreceptors are innervated by the aortic nerve, which then combines with the vagus nerve (X cranial nerve) traveling to the NTS. The NTS modulates the activity of sympathetic and parasympathetic (vagal) neurons in the medulla, which in turn regulate the autonomic control of the heart and blood vessels.

Of these two sites for arterial baroreceptors, the carotid sinus is quantitatively the most important for regulating arterial pressure. The carotid sinus receptors respond to pressures ranging from 60-180 mmHg Receptors within the aortic arch have a higher threshold pressure and are less sensitive than the carotid sinus receptors. Maximal carotid sinus sensitivity occurs near the normal mean arterial pressure; therefore, very small changes in arterial pressure around this “set point” can dramatically alter receptor firing so that autonomic control can be reset in such a way that the arterial pressure remains very near to the set point. This set point changes during exercise, hypertension, and heart failure. The changing set point can explain how arterial pressure can remain elevated during exercise or chronic hypertension.

Barareceptors are sensitive to the rate of pressure change as well as to the steady or mean pressure as well as changes in temperature. Therefore, at a given mean arterial pressure, decreasing the pulse pressure (systolic minus diastolic pressure) decreases the barareceptor firing rate. This can be important during conditions such as hemorrhagic shock in which pulse pressure as well as mean pressure decreases. The combination of reduced mean pressure and reduced pulse pressure reinforces the barareceptor reflex.

An explanation for how the baroreceptors respond to a sudden decrease in arterial pressure and how cardiovascular function is altered is as follows: A decrease in arterial pressure (mean, pulse or both) results in decreased baroreceptor firing. The “cardiovascular center” within the medulla responds by increasing sympathetic outflow and decreasing parasympathetic (vagal) outflow. Under normal physiological conditions, baroreceptor firing exerts a tonic inhibitory influence on sympathetic outflow from the medulla. Therefore, acute hypotension results in a dis-inhibition of sympathetic activity within the medulla, so that sympathetic activity increases. These autonomic changes cause vasoconstriction (increased systemic vascular resistance, SVR), tachycardia and positive inotropy. The latter two changes increase cardiac output. The increases in cardiac output and SVR lead to a partial restoration of arterial pressure.

Baroreceptors adapt to chronic changes in arterial pressure. For example, if arterial pressure suddenly falls when a person stands, the baroreceptor firing rate will decrease; however, after a period of time, the firing returns to near normal levels as the receptors adapt to the lower pressure. Therefore, the long-term regulation of arterial pressure requires activation of other mechanisms (primarily hormonal and renal) to maintain normal blood pressure.

Therapeutic Methods

Also provided is a process of treating spots on the body of a subject in need of an administration of a therapeutically effective amount of temperature stimulation, pressure stimulation, vibration stimulation, or ultrasonic stimulation via a device or system described herein, so as to treat a disease, disorder, or condition, such as hypertension.

Clinical effects of the above-described therapeutic methods of application of a cold/pressure stimulus to the carotid body sinus which upon stimulated causes a strong parasympathetic stimulus through the vagus nerve. The stimulus is in opposition to the sympathetic tone, a counterbalancing effect to decrease heart rate and blood pressure. For example, repeated daily stimulus for a predetermined time, e.g., 5 minutes, causes a continued and stronger suppression of sympathetic tone. It is postulated that this continued decreased sympathetic tone is an adjustment at the hypothalamic level and may have other medical benefits including:
Decreased anxiety
Decreased respiratory rate
Decreased muscle tone and relaxation
Improved mental focus
Decreased inattention ADD

In a recent (May 2011) meta-analysis that included 464,000 people, the authors showed that for a reduction of 10 mmHg systolic or 5 mmHg diastolic, there was a 22% reduction in coronary heart disease events and a 41% reduction in stroke.

Methods described herein are generally performed on a subject in need thereof. A subject in need of the therapeutic methods described herein can be a subject having, for example, been diagnosed with, suspected of having, or at risk for developing hypertension. Hypertension, or high blood pressure, is understood to be a chronic medical condition in which the blood pressure in the arteries is elevated. Blood pressure is conventionally described according to two measurements, systolic and diastolic, which depend on whether the heart muscle is contracting (systole) or relaxed between beats (diastole) and equate to a maximum and minimum pressure, respectively. Normal blood pressure at rest is within the range of about 100 to about 140 mm Hg systolic (top reading) and about 60 to about 90 mm Hg diastolic (bottom reading). A diagnosis of high blood pressure can occur, for example, where blood pressure is at or above about 140/90 mm Hg.

Generally, a safe and effective amount of temperature stimulation, pressure stimulation, vibration stimulation, or ultrasonic stimulation is, for example, that amount that would cause the desired therapeutic effect in a subject while minimizing undesired side effects. In various embodiments, an effective amount of temperature stimulation, pressure stimulation, vibration stimulation, or ultrasonic stimulation described herein can substantially reset balance in the autonomic system, decrease blood pressure, or limit the development of hypertension.

According to the methods described herein, administration can be via contact of a device or system described herein with a spot on the body of the subject. The spot on the body of the subject can be an external tissue of the subject. For example, the spot on the body of the subject can be the carotid artery baroreceptor reflex of the subject. The carotid artery baroreceptor reflex is understood to be located at the left side or right side (carotid sinus) of the subject's neck.

Contact time between a spot on the body of the subject and one or more contact tips of a device or system described herein can be about 30 seconds or more. For example, contact time can be at least about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes and more. For example, contact time can be at from about 1 minute to about 15 minutes. It is understood that recitation of this range of values also refers to each separate range falling within the larger range.

Therapeutic application of temperature stimulation, pressure stimulation, vibration stimulation, or ultrasonic stimulation can be administered, at a reasonable benefit/risk ratio applicable to any medical treatment, in a sufficient amount to decrease blood pressure or limit development of hypertension.

It will be appreciated by those skilled in the art that the contact time of an individual treatment session need not in itself constitute a therapeutically effective amount, as the necessary therapeutically effective amount could be reached by administration of a number of treatment sessions.

The specific therapeutically effective amount of stimulation for any particular subject will depend upon a variety of factors including the disorder being treated and the severity of the disorder; activity of the specific stimulus employed; the age, body weight, general health, sex and diet of the subject; the time of administration; the location of administration; the duration of the treatment; drugs used in combination or coincidental with stimulus employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Shawl (2004) Applied Biopharmaceutics & Pharmacokinetics, McGraw-Hill/Appleton & Lange, ISBN 0071375503). For example, it is well within the skill of the art to start contact time at levels lower than those required to achieve the desired therapeutic effect and to gradually increase the contact time until the desired effect is achieved. If desired, the effective contact time may be divided into multiple sessions for purposes of administration. Consequently, single sessions may contain such amounts or submultiples thereof to make up a therapeutic amount of stimulus. It will be understood, however, that the total contact time from a device of the present disclosure can be decided by an attending physician within the scope of sound medical judgment.

Again, each of the states, diseases, disorders, and conditions, described herein, as well as others, can benefit from contact time from a device or a method described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of temperature stimulation, pressure stimulation, vibration stimulation, or ultrasonic stimulation via a device or system described herein can occur as a single event or over a time course of treatment. For example, temperature stimulation, pressure stimulation, vibration stimulation, or ultrasonic stimulation can be administered one or more times daily, weekly, bi-weekly, or monthly, or a combination thereof. For treatment of acute conditions, the time course of treatment will usually be at least several days. Certain conditions could extend treatment from several days, weeks, months, years, or decades. For example, treatment could extend aver one week, two weeks, or three weeks. For more chronic conditions, treatment could extend from several weeks to several months or even a year or more.

Treatment in accord with devices or methods described herein can be performed prior to, concurrent with, or after conventional treatment modalities for hypertension.

Temperature stimulation, pressure stimulation, vibration stimulation, or ultrasonic stimulation can be administered via a device or system described herein simultaneously or sequentially with another agent, such as an antibiotic, an anti-inflammatory, or another agent. For example temperature stimulation, pressure stimulation, vibration stimulation, or ultrasonic stimulation can be administered simultaneously with another agent, such as an antibiotic or an anti-inflammatory or other anti-hypertensive agents.

### EXAMPLES

The following non-limiting examples are provided to further illustrate the aspects of the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

The following is based, at least in part, on the discovery that regular or periodic lowering of the blood pressure results in training the hypertensive subject's blood pressure to be maintained at a permanently lowered state between therapy sessions as long as a regular or periodic schedule of therapy is maintained.

### Example 1

### Subjects

A medical doctor, M.D. (Staff Cardiologist) measured BP of hypertension patient (RN/VA) prior to device treatment (130/80). Then she applied the PhysioCue device to her right side carotid sinus point for 5 minutes. Her BP was then measured immediately after treatment, and also and 30 minutes later. Her BP readings were: Initial 130/80 pre-treatment; 110/60 immediately post-treatment; 15 min post-treatment was 108/62; 30 min post-treatment was 108/70. Final BP 110/60 approximately 45 min post-treatment.

### Example 2

### Subjects

To select the HDD group and the control group, the standard (see Table 1) defined by JNC-VI (1997) and WHO/ISH (1999) was applied, and persons who had blood pressure exceeding the normal range were prescribed as hypertensive patients.

TABLE 1

| Blood | | Pre- | | | |
|---|---|---|---|---|---|
| Pressure | Normal | hypertension | Stage 1 | Stage 2 | Stage 3 |
| Systolic | <130 | 130-139 | 140-159 | 160-179 | >180 |
| mmHg kPa | <17.3 | 17.3-18.6 | 18.7-21.2 | 21.3-23.9 | >24.0 |
| Diastolic | <85 | 85-89 | 90-99 | 100-109 | >110 |
| mmHg kPa | <11.3 | 11.3-11.9 | 12.0-13.2 | 13.3-14.5 | >14.7 |

To observe a hypertension descending effect of an embodiment of the device described herein, 120 hypertensive patients were divided into an HDD group and a control group without regard to age and sex distinction. The HDD group, which used the hypertension descending device, had 60 members, and the control group, which used antihypertensive agent, also had 60 members.

The members of the HDD group and the control group were selected on the basis of patients who were in similar age and disease condition and the same sex distinction. Moreover, the patients, who participated in this experimental test, were all adults and had blood pressure exceeding normal range, which was measured three times within a predetermined period of time.

Treatment

The HDD group did not take antihypertensive agent at all during the experimental duration, and used only the hypertension descending device. The contact tip of the hypertension descending device was in contact with the patient's carotid sinus located at the left side or the right side of the human body's neck portion.

Blood pressure was measured with an automatic blood pressure measuring device (JAWON Medical Co., Ltd.).

The experimental duration was about 3 months for about 5 minutes every day, and during the above duration, blood pressure was measured before treatment, directly after the treatment, and 30 minutes after treatment.

The control group took oral antihypertensive agent according to a physician's prescription. The experimental duration was about 3 months, and blood pressure was measured once every day.

During the experimental duration, the members of the HDD group did not take antihypertensive agent at all, and used only the hypertension descending device manufactured by the JAWON Medical Co., Ud. On the other side, the members of the control group took antihypertensive agent.

Effect Criterion

A criterion of treatment effect was divided into three stages according to the descending level of systolic blood pressure, and the treatment effect was judged by percentage.
A. Excellent: in case of that systolic blood pressure more than 20 mmHg was descended.
B. Good: in case of that systolic blood pressure of about 10-20 mmHg was descended.
C. Poor: in case of that systolic blood pressure of about 10 mmHg or less was descended.

Results

The subject members for study were all 120 people, including the HDD group having 60 members and the control group having 60 members. The HDD group had 25 males and 35 females, and its average age was 58.9 (see e.g., TABLE 2).

TABLE 2
Distribution of Age in HDD and Control Group

| Age | Male N. | Female N. | Total |
|---|---|---|---|
| 40-49 | 5 | 5 | 10 |
| 50-59 | 7 | 14 | 21 |
| 60-69 | 12 | 16 | 28 |
| 70-79 | 1 | 0 | 1 |
| Total | 25 | 35 | 60 |

N: Number of patient

The control group had 60 members including 28 males and 32 females, and its average age was 58.2 (see e.g., TABLE 3).

TABLE 3
Distribution of Sex in HDD and Control Group

| Age | Male N. | Female N. | Total |
|---|---|---|---|
| 40-49 | 2 | 4 | 6 |
| 50-59 | 14 | 16 | 30 |
| 60-69 | 12 | 12 | 24 |
| 70-79 | 0 | 0 | 0 |
| Total | 28 | 32 | 60 |

The 60 members of the HDD group were classified into 18 persons of hypertension stage 1; 37 persons of hypertension stage 2; and 5 persons of hypertension stage 3. The 60 members of the control group were classified into 18 persons of hypertension stage 1; 32 persons of hypertension stage 2; and 10 persons of hypertension stage 3 (see e.g., TABLE 4).

TABLE 4
Grade Distribution of Hypertention

| | HDD | Control Group | Total |
|---|---|---|---|
| Hypertention Grade 1 | 18 | 18 | 36 |
| Hypertention Grade 2 | 37 | 32 | 69 |
| Hypertention Grade 3 | 5 | 10 | 15 |
| Total | 60 | 60 | 120 |

Treatment Effect of Hypertension Stages 1 and 2.

Results showed that in the HDD group, the hypertensive patients of hypertension stages 1 and 2 were total 55 persons, wherein 25 persons (45.5%) obtained an excellent curing effect, 23 persons (41.8%) obtained a good curing effect, and so, total 48 persons (87.3%) obtained an effective curing treatment result, but only 7 persons (12.7%) obtained a poor curing treatment result (see e.g., TABLE 5).

Meanwhile, in the control group, the hypertensive patients of hypertension stages 1 and 2 were total 50 persons, wherein 23 persons (46.0%) obtained an excellent curing treatment effect after taking oral antihypertensive agent, 21 persons (42.2%) obtained a good curing treatment effect, and so, total 44 persons (88.0%) obtained an effective curing treatment result, but only 6 persons (12.0%) obtained a poor curing treatment result (see e.g., TABLE 5).

TABLE 5
Result in hypertention grade 1 and 2

| | HDD | | Control Group | |
|---|---|---|---|---|
| Excellent | 25 | (45.5%) | 23 | (46.0%) |
| Good | 23 | (41.8%) | 21 | (42.0%) |
| Poor | 7 | (12.7%) | 6 | (12.0%) |
| Total | 55 | | 50 | |
| Effect | 48/55 | (87.3%) | 44/50 | (88.0%) |

In case of the hypertension stages 1 and 2, there was little difference between the HDD group and the control group.

Treatment Effect of Hypertension Stage 3.

There were 5 hypertensive patients of hypertension grade 3 in the HDD group, wherein the 5 hypertensive patients of hypertension stage 3 obtained little curing treatment effect by the hypertension descending device as blood pressure descending of the 5 hypertensive patients was about 10 mmHg or less.

There were 10 hypertensive patients of hypertension stage 3 in the control group, wherein 7 persons (70%) of the hypertensive patients obtained an excellent curing treatment effect after taking antihypertensive agent, and 3 persons (30%) obtained a poor curing effect (see e.g., TABLE 6).

TABLE 6
Result in Hypertention Grade 30

| | HDD | | Control Group | |
|---|---|---|---|---|
| Excellent | 0 | (0%) | 7 | (70%) |
| Good | 0 | (0%) | 0 | (0%) |
| Poor | 5 | (100%) | 3 | (30%) |
| Total | 5 | | 10 | |

Comparison of Treatment Effect.

48 persons (80.0%) of the total 60 hypertensive patients in the HDD group obtained an effective curing treatment result using the hypertension descending device according to the present invention, but 51 persons (85.0%) in the control group obtained the effective curing treatment result after taking hypertensive agent (see e.g., TABLE 7).

TABLE 7
Comparison of Effect between HDD and Control Group

| | HDD | Control Group |
|---|---|---|
| Effect | 48/60 (80.0%) | 51/60 (85.0%) |

Conclusion.

As described above an embodiment device of the present disclosure applied cold stimulation to the carotid sinus of 120 hypertensive patients of hypertension stages 1, 2 and 3. A summary of above results are as follows.
1. 87.3% of the hypertensive patients of hypertension stages 1 and 2 in the HDD group obtained the effective curing treatment result by the hypertension descending device according to the present invention, and 88.0% of the hypertensive patients of hypertension stages 1 and 2 in the control group obtained the effective curing treatment result after taking antihypertensive agent, and so, the HDD group and the control group showed similar curing treatment effect.
2. The curing treatment effect of the hypertensive patients of hypertension stage 3 in the HDD group was stili less than that of the hypertensive patients of hypertension stage 3 in the control group.
3. The effective curing treatment result of the total hypertensive patients in the HDD group was 80.0%, but that of the total hypertensive patients in the control group was 85.0%.

As shown above, a device of the present disclosure can be used as adjunct or substitution treatment for antihypertensive agent for the hypertensive patients of hypertension stages 1 and 2, and help to prevent the progress from the hypertension stage 1 to the hypertension stage 2 or 3.

Migraines and Other Headaches

The method may be directed to treating headaches, e.g., improving palliative symptoms, reducing or eliminating the cause of the headache, achieving an effective cure, etc. In some aspects, the device (not shown) can include one or a combination of the following: a heat sink (e.g., a metal heat sink) with a good heat conductivity, a conical cooling pin having a number of miniaturized vibration motors arranged at regular intervals to radiate or remove heat to the outside efficiently, a coupling slit formed in the front end thereof to couple a cap, or through holes formed in the side surface to draw out electric wires.

A contact tip can protrude an upper end portion of a case so as to be in contact with a point on the body of the subject and the thermoelectric Peltier module can be disposed on a lower end of the contact tip to cool down the temperature of the contact tip to the temperature set by the user. In some embodiments, a thermoelectric Peltier module can be mounted on the heat sink where a heat generating surface of the thermoelectric Peltier module is directed to the heat sink, or a cooling surface of the thermoelectric Peltier module can have a contact tip, which can have a hemispheric magnet mounted at the top of the thermoelectric Peltier module. The thermocouple electric wires of the thermoelectric module can be drawn out of the lower end of the heat sink through the holes formed in the side surface of the heat sink. The contact tip can have an optimal volume to transmit cooling temperature (0° C.±5° C.), which can be set by a user or predetermined by a manufacturer, to a contact point on the body of a subject. A cooling fan can be mounted on or near the heat sink (e.g., at the lower end portion of the heat sink) to effectively remove heat emitted from the heat generating surf ace of the thermoelectric module, thereby maximizing a cooling efficiency of the thermoelectric module. One skilled in the art will recognize that the device, its components, and use of the device may be effected using various components or combination of components, various and or multiple application sites, e.g., cranial, temporal, a range of application times, e.g., 15 seconds to 10 minutes, 1 minute to 7 minutes, etc., and application may be repeated at various intervals for durations of time, e.g., intervals of once every 30 minutes for an hour, once every 15 minutes for half of an hour, etc.

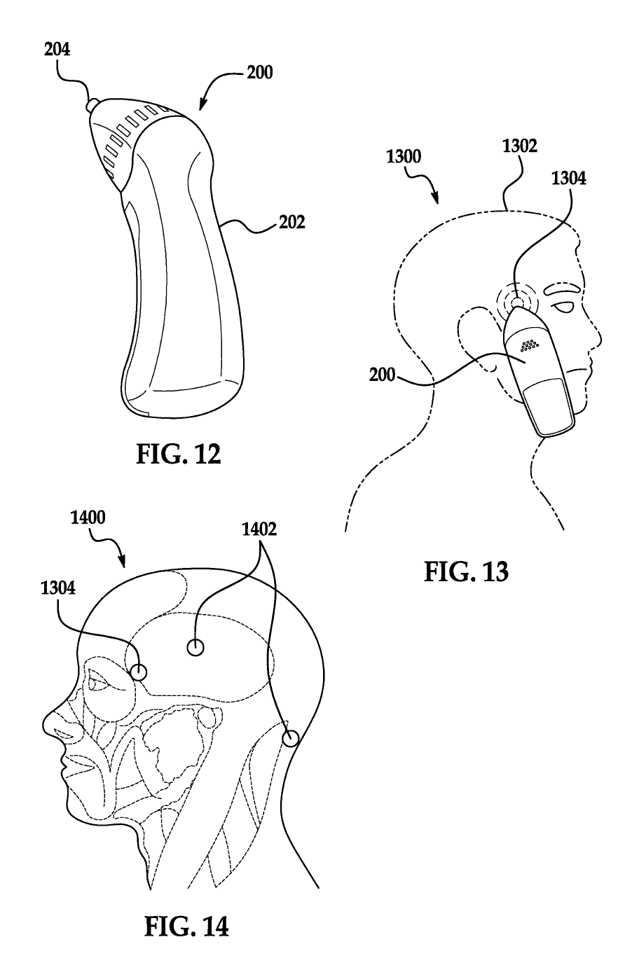

For example, and with reference to FIGS. 12 and 13, there are shown a subject 1202 and corresponding treatment sites. In FIG. 12, the device 202 having a contact tip 204 is applied to an application site in a temporal area 1204. The corresponding general sub-tissue region is shown in FIG. 13, where portions of the corresponding musculoskeletal structures in the region are depicted.

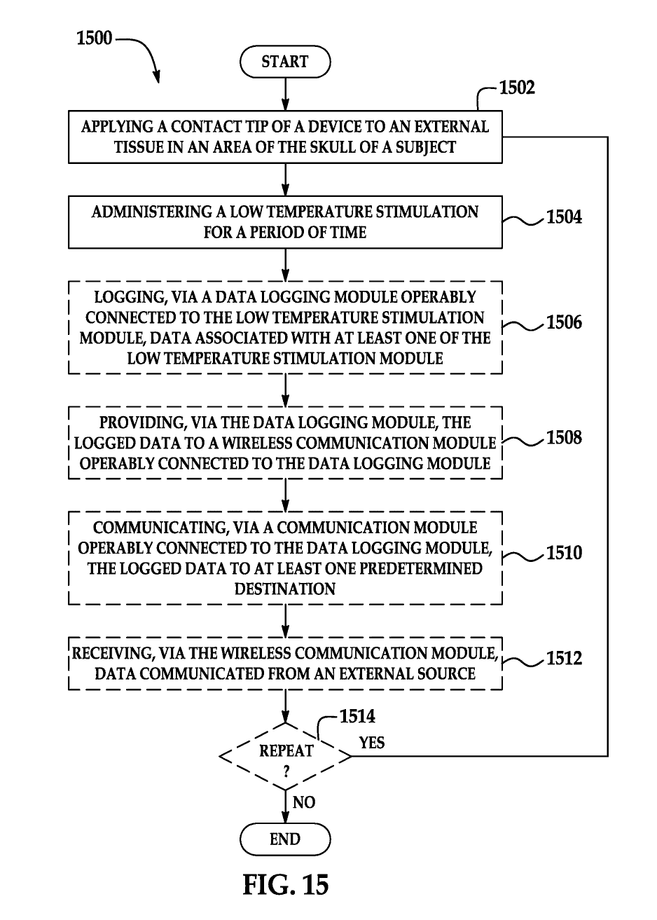

In various aspects and with reference to FIGS. 15-17, the method may be directed to treating a headache and include the following steps in various combinations. Optional and/or alternative steps are shown in phantom outline. For example, FIG. 15 shows a flow diagram of a method 1500 of treating a headache having a step 1502 applying a contact tip of a device to an external tissue in an area of the skull of a subject; and step 1504 administering a low temperature stimulation for a period of time, wherein, the contact tip of the device is operably connected with a case and a low temperature stimulation module such that the contact tip provides low temperature stimulation. The external tissue in an area of the skull of a subject may comprise a predetermined application site on the skull, e.g., a cranial site or a temporal site.

In certain embodiments, the temperature stimulation module may effects a 5 contact tip temperature of about −5° C. to about 5° C. Other ranges are contemplated, e.g., −2° C. to about 2° C., −10° C. to about 10° C., −15° C. to about 15° C., etc. As previously discussed, the low temperature stimulation module may comprise one or more of a thermoelectric module; a temperature sensor, a temperature selector, or a temperature controller. The device may comprises a temperature adjustment the temperature adjustment module comprises a dial.

The method 1500 may further consist of one or more of the following: step 1506 logging, via a data logging module operably connected to the low temperature stimulation module, data associated with at least one of the low temperature stimulation module, step 1508 providing, via the data logging module, the logged data to a wireless communication module operably connected to the data logging module; and step 1510 communicating, via the wireless communication module operably connected to the data logging module, the logged data to at least one predetermined destination. As previously discussed, the predetermined destination may comprise at least one of a remote data server and a mobile device.

Further, the method may consist of one or more of the following: step 1512 receiving, via the wireless communication module, data communicated from an external source and step 1514 repeating, for a predetermined number of times and at a predetermined interval, any combination of the preceding steps.

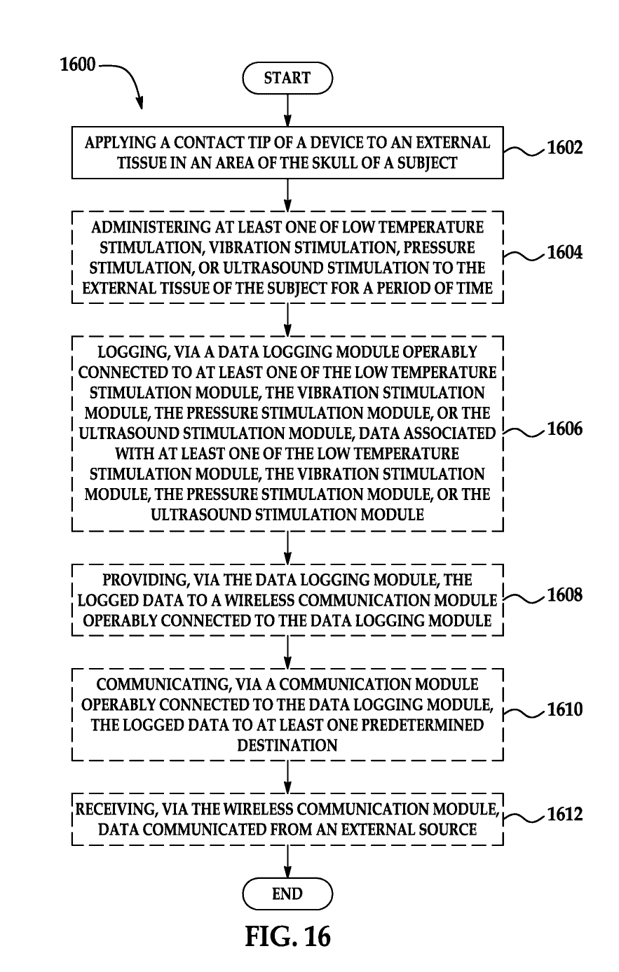

FIG. 16 is a flow diagram of a method 1600 of treating a headache consisting of one or more of: the step 1602 applying a contact tip of a device to an external tissue in an area of the skull of a subject; and step 1604 administering at least one of low temperature stimulation, vibration stimulation, pressure stimulation, or ultrasound stimulation to the external tissue of the subject for a period of time. In various aspects, the device comprises the contact tip operably connected with a case; a low temperature stimulation module; and at least one of a vibration stimulation module, a pressure stimulation module, or an ultrasound stimulation module; wherein, the contact tip is suitable for contact with an external tissue of a subject; the contact tip is operably connected to the low temperature stimulation module such that the contact tip provides low temperature stimulation; and the contact tip is operably connected to the vibration stimulation module, the pressure stimulation module, or the ultrasound stimulation module, such that the contact tip provides vibration stimulation, pressure stimulation, or ultrasound stimulation, respectively, wherein the device comprises the contact tip operably connected with a case; a low temperature stimulation module; and at least one of a vibration stimulation module, a pressure stimulation module, or an ultrasound stimulation module; wherein,

the contact tip is suitable for contact with an external tissue of a subject;

the contact tip is operably connected to the low temperature stimulation module such that the contact tip provides low temperature stimulation; and

the contact tip is operably connected to the vibration stimulation module, the pressure stimulation module, or the ultrasound stimulation module, such that the contact tip provides vibration stimulation, pressure stimulation, or ultrasound stimulation, respectively.

In various aspects, the external tissue in an area of the skull of a subject comprises a predetermined application site on the skull, e.g., a cranial site, a temporal site, etc.

In various aspects, the method 1600 may further consist of one or more of the following: step 1606 logging, via a data logging module operably connected to at least one of the low temperature stimulation module, the vibration stimulation module, the pressure stimulation module, or the ultrasound stimulation module, data associated with at least one of the low temperature stimulation module, the vibration stimulation module, the pressure stimulation module, or the ultrasound stimulation module; step 1608 providing, via a data logging module, the logged data to a wireless communication module operably connected to the data logging module; step 1610 communicating, via a wireless communication module operably connected to the data logging module, the logged data to at least one predetermined destination; and step 1612 receiving, via the wireless communication module, data communicated from an external source.

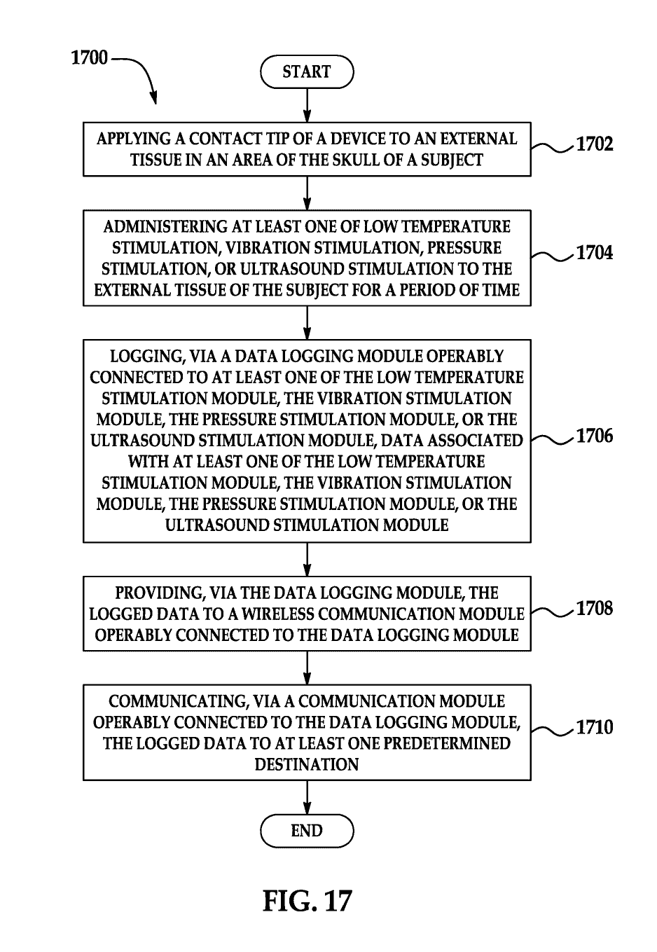

FIG. 17 shows a flow diagram of a method 1700 of treating a headache, the method consisting of step 1702 applying a contact tip of a device to an external tissue in an area of the skull of a subject; step 1704 administering at least one of low temperature stimulation, vibration stimulation, pressure stimulation, or ultrasound stimulation to the external tissue of the subject for a period of time; step 1706 logging, via a data logging module operably connected to at least one of the low temperature stimulation module, the vibration stimulation module, the pressure stimulation module, or the ultrasound stimulation module, data associated with at least one of the low temperature stimulation module, the vibration stimulation module, the pressure stimulation module, or the ultrasound stimulation module; step 1708 providing, via the data logging module, the logged data to a wireless communication module operably connected to the data logging module; and step 1710 communicating, via the wireless communication module operably connected to the data logging module, the logged data to at least one predetermined destination, wherein the device comprises the contact tip operably connected with a case; a low temperature stimulation module; and at least one of a vibration stimulation module, a pressure stimulation module, or an ultrasound stimulation module; wherein, the contact tip is suitable for contact with an external tissue of a subject; the contact tip is operably connected to the low temperature stimulation module such that the contact tip provides low temperature stimulation; and the contact tip is operably connected to the vibration stimulation module, the pressure stimulation module, or the ultrasound stimulation module, such that the contact tip provides vibration stimulation, pressure stimulation, or ultrasound stimulation, respectively, wherein the device comprises the contact tip operably connected with a case; a low temperature stimulation module; and at least one of a vibration stimulation module, a pressure stimulation module, or an ultrasound stimulation module; wherein, the contact tip is suitable for contact with an external tissue of a subject; the contact tip is operably connected to the low temperature stimulation module such that the contact tip provides low temperature stimulation; and the contact tip is operably connected to the vibration stimulation module, the pressure stimulation module, or the ultrasound stimulation module, such that the contact tip provides vibration stimulation, pressure stimulation, or ultrasound stimulation, respectively.

As these and other variations and combinations of the features discussed above can be utilized without departing from the subject matter defined by the claims, the foregoing description of the embodiments should be taken by way of illustration rather than by way of limitation of the subject matter defined by the claims. As an example, the preceding method steps do not have to be performed in the precise order described above. Rather, various steps can be handled in a different order or simultaneously. Steps can also be omitted unless otherwise stated. In addition, the provision of the examples described herein, as well as clauses phrased as “such as,” “including” and the like, should not be interpreted as limiting the subject matter of the claims to the specific examples; rather, the examples are intended to illustrate only one of many passible embodiments. Further, the same reference numbers in different drawings can identify the same or similar elements.

1. (canceled)

2. The device of claim 21, wherein the temperature stimulation module effects a contact tip temperature of about −10° C. to about 10° C.

3. The device of claim 21, wherein the low temperature stimulation module comprises one or more of a thermoelectric module; a temperature sensor, a temperature selector, or a temperature controller.

4. The device of claim 21, further comprising a temperature adjustment module.

5. The device of claim 4, wherein the temperature adjustment module comprises a digital dial.

6. The device of claim 21, further comprising a data logging module and a wireless communication module;
wherein at least one of the low temperature stimulation module, the vibration stimulation module, the pressure stimulation module, or the ultrasound stimulation module is operably connected to the data logging module; and
the data logging module is operably connected to the wireless communication module.

7. The device of claim 21, further comprising one or more of a temperature monitoring module, a blood pressure monitoring module, a pulse oximetry monitoring module, a glucose monitoring module, a heart rate monitoring module, a navigation module, and a scanning module.

8.-11. (canceled)

12. The method of claim 24, further comprising:
logging, via a data logging module operably connected to at least one of the low temperature stimulation module, the vibration stimulation module, the pressure stimulation module, or the ultrasound stimulation module, data associated with at least one of the low temperature stimulation module, the vibration stimulation module, the pressure stimulation module, or the ultrasound stimulation module.

13. The method of claim 24, further comprising:
providing, via the data logging module, the logged data to a wireless communication module operably connected to the data logging module; and
communicating, via the wireless communication module operably connected to the data logging module, the logged data to at least one predetermined destination.

14. The method of claim 13, wherein the predetermined destination comprises at least one of a remote data server and a mobile device.

15. The method of claim 24, further comprising a step of receiving, via the wireless communication module, data communicated from an external source.

16. The method of claim 24, wherein the data comprises at least one of low temperature stimulation data, blood pressure data, heart rate data, spatial position with respect to the subject data, operation time data, tip-tissue contact time data, human body temperature data, pulse oximetry data, or scanned information.

17. The method of claim 24, wherein the step of applying a contact tip of a device to an external tissue of a subject further consists of applying a contact tip of a device to an external tissue of a subject for period of time comprising approximately 1 minute to 10 minutes.

18. The method of claim 24, further consisting of the step of repeating, for a predetermined number of times and at predetermined intervals of between 15 minutes and 30 minutes, the steps of claim 24.

19. The method of claim 18, wherein step of repeating, for a predetermined number of times and at a predetermined interval, the steps of claim 24 results in consistently lower blood pressure of the subject when the consistently lower blood pressure is compared to the subject's blood pressure prior to a first application of the method to the subject.

20. (canceled)

21. A device comprising:
a case;
a contact tip operably connected with the case; and
a low temperature stimulation module operably connected to the contact tip;
wherein the contact tip is shaped to be in contact with external tissue of a subject;
wherein the contact tip is operably connected to the low temperature stimulation module such that the contact tip provides low temperature stimulation to the subject.

22. The device of claim 21 further comprising:
a vibration stimulation module operably coupled to the contact tip, such that the contact tip provides vibration stimulation in addition to the low temperature stimulation.

23. The device of claim 21 further comprising:
at least one additional module comprising one or both of a pressure stimulation module and/or an ultrasound stimulation module, the at least one additional module operably connected to the contact tip, such that the contact tip provides pressure stimulation and/or ultrasound stimulation.

24. A method for stimulating external tissue of a subject to reduce hypertension, the method comprising:
applying a contact tip of a device to the external tissue of a subject; and
administering a low temperature stimulation to the external tissue of the subject for a period of time,
wherein the device comprises the contact tip operably connected with a case and a low temperature stimulation module; and
wherein the contact tip is suitable for contact with the external tissue of the subject; and
wherein the contact tip is operably connected to the low temperature stimulation module such that the contact tip provides low temperature stimulation.

25. The method of claim 24 wherein the contact tip is operably connected to a vibration stimulation module and/or a pressure stimulation module, such that the contact tip provides vibration stimulation and/or pressure stimulation, respectively.

26. The method of claim 19, wherein the step of applying a contact tip of a device to an external tissue of a subject further consists of applying a contact tip of a device to an external tissue of a subject for period of time comprising approximately 1 minute to 10 minutes.
